(19) United States
(12) Patent Application Publication
(10) Pub. No.: US 2021/0283341 A1
(43) Pub. Date: Sep. 16, 2021

(54) AN INJECTION DEVICE WITH AN IMMOBILIZATION MECHANISM

(71) Applicant: Novo Nordisk A/S, Bagsvaerd (DK)

(72) Inventors: Niels Frederik Keiser-Nielsen, Niva (DK); Emma Katherine Goodwin, Alleroed (DK)

(21) Appl. No.: 17/258,205

(22) PCT Filed: Jul. 5, 2019
(86) PCT No.: PCT/EP2019/068084

§ 371 Date: Jan. 6, 2021

(30) Foreign Application Priority Data
Jul. 12, 2018 (EP) 18183063.9

(51) Int. Cl. A61M 5/32

(52) U.S. Cl. CPC A61M 5/326; A61M 2005/3247

(57) ABSTRACT

The invention relates to an injection device for injecting doses of a liquid drug, which comprises an immobilization mechanism such that the further use of the injection device is immobilized in response to the needle cannula (20) being exposed to an axial force above a predetermined value: the immobilization mechanism comprises a flexible arm (50) which apply a radial force onto the needle cannula at least during injection.

### THE TECHNICAL FIELD OF THE INVENTION

The invention relates to a shielded injection device wherein a movable needle shield covers the distal tip of the needle cannula between injections. The invention especially relates to an immobilization mechanism for such injection device.

### DESCRIPTION OF RELATED ART

Injection devices wherein the distal tip of the needle cannula is maintained inside a cleaning chamber carried by a movable needle shield between injections are e.g. known from WO 2017/032599 and from WO 2017/084976. In one example the cleaning agent present inside the cleaning chamber is a volume of the liquid drug contained in the injection device. Since a liquid drug often contains a preservative the presence of this preservative cleans the distal tip of the needle cannula between injections.

In the injection device disclosed in WO 2017/032599, the needle shield is able to move helically upon rotation of the needle shield. The needle shield thus move in the proximal direction upon rotation such that the distal tip of the needle cannula protrudes into a position distally outside the cleaning chamber as depicted in FIG. 11 of WO 2017/032599. In this position, often referred to as the NPR (Needle Pressure Relief) position, the liquid system is vented and the needle shield is unlocked such that the needle shield can perform an axial movement when pressed against the skin of the user to perform an injection.

The injection device disclosed in WO 2017/084976 is further provided with a dynamic immobilization mechanism which is able to immobilize the injection device if the needle cannula is exposed to a force above a predetermined value. Should the user e.g. drop the injection device onto a hard surface such that the exposed needle cannula is exposed to a high force then a mechanism inside the housing will be activated and lock the needle shield from further movement. The immobilization mechanism is based on an axial movement of the needle hub upon impact. When the distal tip of the needle cannula is exposed to a high force, then both the needle cannula and the needle hub to which the needle cannula is attached is moved in the proximal direction. This proximal movement releases a locking element which locks the needle shield from further movement.

However, a requirement for this immobilization mechanism is that the needle hub is able to move axially upon impact which again requires very narrow tolerances and also a risk, depending on the tolerances, that the needle hub moves axially unintended which would thus lock the needle shield.

A more static immobilization mechanism is disclosed in U.S. Pat. No. 7,597,684, which discloses an injection device in which the user following an injection manually can operate a radial working element which is able to bend the needle cannula. During bending of the needle cannula, the distal tip of the needle cannula retracts into the housing thus immobilizing the injection device.

### DESCRIPTION OF THE INVENTION

It is an object of the present invention to provide an injection device with an immobilization mechanism which depend less on tolerances.

Accordingly, in one aspect of the present invention, an injection device is provided which has an immobilization mechanism. The injection device can be used for injecting one or more doses which doses can be of a predetermined size or individually set by the user when preparing an injection.

The injection device comprises a housing structure which secures a container such as a cartridge containing the liquid drug to be injected. The liquid drug is preferably a preservative containing liquid drug.

Further, a needle cannula is connected to the housing structure. Such needle cannula usually has a distal end with a distal tip for penetrating the skin of a user during injection and a proximal end for connecting to the container and a lumen there between for creating a liquid communication between the container and the user.

In one embodiment, the needle cannula is secured in a needle hub which is attached to the housing structure.

At least partly surrounding the needle cannula a movable needle shield is provided. This movable needle shield is movable e.g. helically relatively to the housing structure between a first position and a second position.
the first position is defined as a position in which the movable needle shield covers at least the distal tip of the needle cannula, wherein covers is meant to be at least in a radial plane, and
the second position is defined as a position in which the needle shield is retracted such that the distal tip of the needle cannula is exposed.

The movable needle shield preferably carries a cleaning chamber containing a cleaning agent suitable for cleaning the distal tip of the needle cannula between injections and the distal tip of the needle cannula is preferably maintained inside the cleaning chamber between injections.

The injection device further comprises an immobilization mechanism which immobilizes the injection device in response to the needle cannula being exposed to an axial force above a predetermined value and which immobilization mechanism comprises at least one flexible arm which applies a radial force onto the needle cannula.

Should the user accidentally drop the injection device onto a hard surface such that the needle cannula is exposed to an axial force above a predetermined value sufficient to damage the needle cannula, the flexible arm applying a radial force onto the needle cannula will response to the axial force and immobilize further use of the injection device.

The flexible arm is thus pre-tensed and urges a radial force onto the needle cannula such that when the needle cannula start to bend, the pre-tensed flexible arm will enforce the bending.

The predetermined value sufficient to damage the needle cannula is the axial force needed to bend the injection cannula. This force obviously depends on the gauge of the needle cannula such that a very thin needle with a large gauge size has a lower bending force than a thicker needle cannula having a smaller gauge size.

Whenever the axial force applied to the needle cannula passes this predetermined value, the needle cannula will start to bend and the radial force applied by the flexible arm will thus support the bending of the needle cannula.

The flexible arm is preferably movable in the radial direction i.e. perpendicular to the lengthwise axis of the injection device and can be moved at least between a first radial position and a second radial position. The radial movement is automatically performed in response to bending of the needle cannula as the flexible arm is pretensed. The flexible arm thus both follows the bending of the needle cannula and amplifies the bending.

Further, when the flexible arm is in its second radial position the flexible arm prevents axial movement of the needle shield. In order to accomplish this, the flexible arm is in one example provided with a distally pointing end surface which engages the needle shield or at least a part of the needle shield when the flexible arm is in the second radial position. The part of the needle shield being engaged can be any part and even a part of the needle shield or even a separate part connected to the needle shield. The needle shield is thus hindered in its axial, and proximal, movement by the flexible arm which thus abuts the needle shield if the user tries to perform an injection by pushing the shield against the skin or otherwise move the needle shield proximally.

The needle shield is thus able to be moved in the proximal direction relatively to the housing structure when the flexible arm is in the first radial position, however, when the flexible arm is positioned in the second radial position the flexible arm hinders movement of the needle shield in the proximal direction.

Both the needle cannula and the flexible arm are further connected to a needle hub. The needle cannula is in a usual manor anchored in an opening in the needle hub whereas the flexible arm is either permanently coupled to the needle hub or in a different example made as a separate part which is attached to the needle hub. In a further example, the flexible arm is manufactured from a metallic tape which is thus bended into its final shape and connected to the needle hub e.g. by a clip or snap function.

The flexible arm is preferably urged radially in to abutment with the needle cannula. This force urging the flexible arm radially can either be a force delivered by a separate resilient element such as a spring or the like, or it can be a force delivered by the flexibility inherent in the flexible arm. In the latter example, the flexible arm is pretensed e.g. by an abutment with another part of the construction.

In one example the flexible arm is provided with an outwardly pointing protrusion which is able to engage with the needle shield to thereby urge the flexible arm radially.

When the flexible arm is radially urged against the needle cannula, the flexible arm will move further radially if the needle cannula is bended. Actually the radial force urging the flexible arm against the needle cannula will enhance the bending of the needle cannula and secure that if the needle cannula is bended it is bended sufficient to the distal tip to be moved inside the cleaning chamber of the needle shield to thereby fully immobilize the injection device

As e.g. disclosed in WO 2017/032599, the needle shield is rotatable relatively to the housing structure from a locked to an unlocked position and the protrusion provided on the flexible arm only abuts the needle shield when the needle shield is rotated to the unlocked position. In such situation, the flexible arm only delivers a radial force onto the needle cannula when the needle shield is rotated to its unlocked position which is the only situation in which the needle cannula can be damaged. When the injection device is locked, the flexible arm is automatically retracted from the needle cannula.

In one specific example the outwardly pointing protrusion provided on the flexible arm is able to engage with a raised locking area provided inside the needle shield such that this engagement urges the flexible arm radially into abutment with the needle cannula.

The flexible arm is henceforth urged radially into abutment with the needle cannula when the needle shield is rotated to unlock the injection device and moved out of engagement with the needle cannula once the user locks the injection device by rotating the needle shield to the locked position.

In order to properly lift the outwardly pointing protrusion on the flexible arm and thus the flexible arm, the raised locking area is preferably provided with a sloped surface which gradually lifts the outwardly pointing protrusion and the flexible arm towards the centre line of the injection device.

In order to secure that the flexible arm properly engages the needle cannula when moved radially, the flexible arm is in one example provided with a radial extension which thus is the part abutting the needle cannula as the flexible arm is moved radially.

Henceforth, when the user rotates the needle shield from its locked to its unlocked position, the needle shield moves helically in the proximal direction whereby the distal tip of the needle cannula enters into the NPR position i.e. a position wherein the distal tip is positioned just distal to the needle shield. However, the needle cannula is not physically moved, only the needle shield is moved. In this NPR position, the flexible arm is pre-tensed by its engagement with the needle shield such that the flexible arm, in the NPR position, apply a radial force onto the needle cannula.

When the injection is finished and the user rotate the needle shield back to its locked position, the pre-tense of the flexible arm is removed and the flexible arm bounces back to its first radial position in which position the flexible arm can be designed such that the flexible arm engages the needle shield in the longitudinal direction.

### Definitions

An “injection pen” is typically an injection apparatus having an oblong or elongated shape somewhat like a pen for writing. Although such pens usually have a tubular cross-section, they could easily have a different cross-section such as triangular, rectangular or square or any variation around these geometries.

The term “Needle Cannula” is used to describe the actual conduit performing the penetration of the skin during injection. A needle cannula is usually made from a metallic material such as e.g. stainless steel but could also be made from a polymeric material or a glass material. The needle cannula can be anchored in a needle hub or directly in the injection device without the use of a needle hub. If the needle cannula is anchored in a needle hub this needle hub can be either permanently or releasable coupled to the injection device.

As used herein, the term “drug” is meant to encompass any drug-containing flowable medicine capable of being passed through a delivery means such as a hollow needle cannula in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

The term “preservative containing liquid drug” is preferably used to describe a liquid drug containing any kind of a preservative. Such liquid drug could in one example be a blood sugar regulating liquid drug such as insulin, insulin analogue, GLP-1 or GLP-2, and the preservative contained in the liquid drug could in one example be phenol, meta-cresol or any combination thereof. However, any kind of preservative can under this term be combined with any kind of liquid drug.

“Cartridge” is the term used to describe the container actually containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

“Cleaning chamber” is in the present description broadly meant to be any kind of reservoir containing a cleaning solvent to clean at least the distal tip of the needle cannula between subsequent injections. Such cleaning chamber is preferably both distally and proximally sealed by a pierceable septum or the like. However, the proximal septum could be replaced by any kind of sealing which would seal against the outer surface of the needle cannula e.g. a movable plunger with some kind of sealing. The distal septum and the proximal septum or seal of the cleaning chamber defines a confinement containing the cleaning solvent which cleaning solvent in a preferred embodiment is identical to the preservatives contained in the liquid drug used in the specific injection device. In a most preferred solution, the same preservative containing liquid drug is present in both the cleaning chamber and in the cartridge of the injection device thereby avoiding contamination of the preservative containing drug inside the cartridge.

By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

The term “Permanently connected” or “permanently embedded” as used in this description is intended to mean that the parts, which in this application is embodied as a cartridge permanently embedded in the housing, requires the use of tools in order to be separated and should the parts be separated it would permanently damage at least one of the parts.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1 show a cross-sectional view of the front end of the injection device with the needle shield in the locked mode.

FIG. 2 show a cross-sectional view of the front end of the injection device with the needle shield in the un-locked mode.

FIG. 3 show a cross-sectional view of the front end of the injection device during injection.

FIG. 4 show a cross-sectional view of the front end of the injection device with the needle cannula bended.

FIG. 5 show a perspective view of the needle hub and the flexible arm.

FIG. 6 show a perspective view of the needle hub and an example of a flexible arm dismounted from the needle hub.

FIG. 7 show a perspective view of the needle hub and an example of a flexible arm mounted on the needle hub.

The figures are schematic and simplified for clarity, and they just show details, which are essential to the understanding of the invention, while other details are left out. Throughout, the same reference numerals are used for identical or corresponding parts.

### DETAILED DESCRIPTION OF EMBODIMENT

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

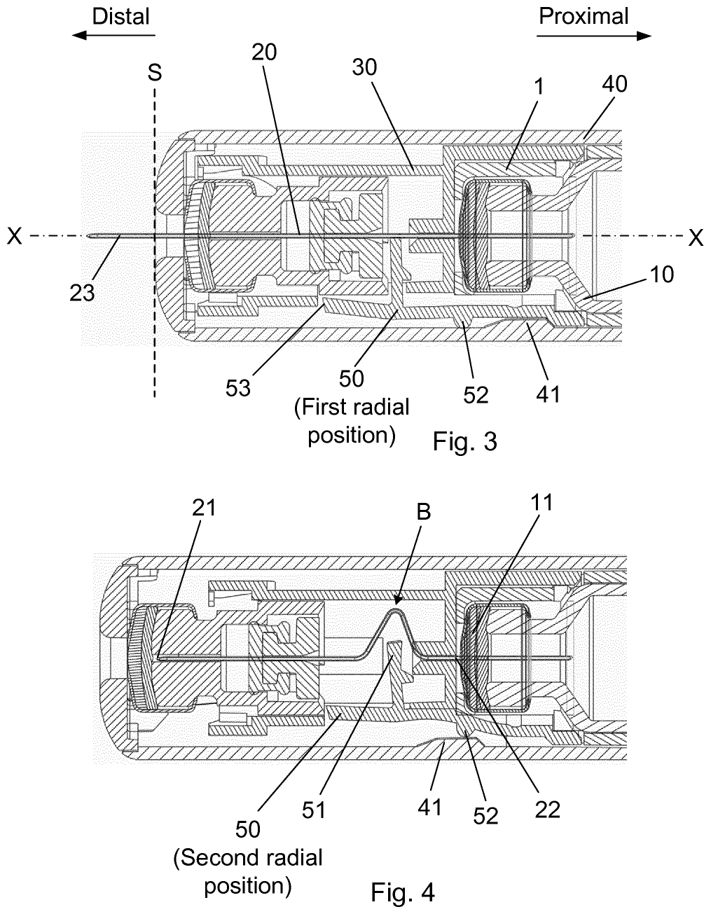

In that context it may be convenient to define that the term “distal end” in the appended figures is meant to refer to the end of the injection device carrying the needle cannula and the tip of the needle cannula doing the actual penetration of the skin of a user, whereas the term “proximal end” is meant to refer to the opposite end. Distal and proximal is meant to be along an axial orientation extending along the central axis (X) of the injection device as also disclosed in FIG. 3.

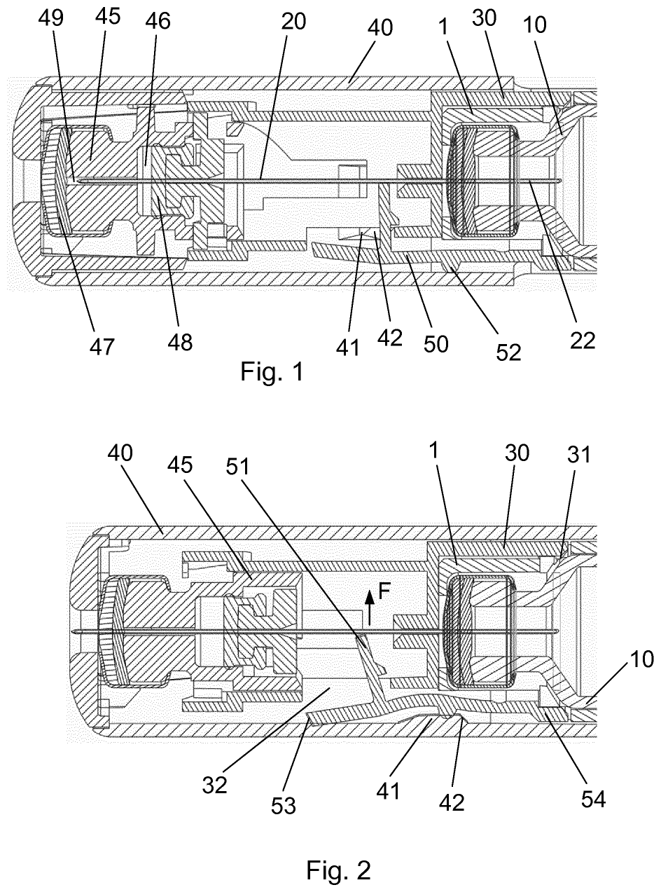

The FIGS. 1 to 4 disclose the front part of the injection device. The dose setting and injection mechanism often referred to as the dose engine is not shown in the figures but could in one example be a torsion spring dose engine as disclosed in WO 2019/002020.

FIG. 1, FIG. 2 and FIG. 3 all disclose the injection device during ordinary use. In FIG. 1 the needle shield 40 is locked in the initial first position. In FIG. 2 the needle shield is still in the first position but has been rotated to the unlocked NPR position and in FIG. 3 the needle shield (40) is pushed in proximal direction into the second position and an injection is being performed.

The liquid drug to be injected is contained in a cartridge 10 which is secured in a housing structure 1. The liquid drug preferably contains a preservative and a volume of the preservative containing liquid drug is also present in a cleaning chamber 46 to clean the distal tip 21 of the needle cannula 20 between injections as will be explained.

The housing structure 1 can be made from several components which a connected to form one housing or alternative the housing structure 1 can be moulded as one unitary part. Usually such housing structure 1 comprises a cartridge holder part which secures the cartridge 10.

During initiation of the injection device a volume of the preservative containing drug which operates as the cleaning agent is pumped from the cartridge 10 and into the cleaning chamber 46 through the lumen 23 of the needle cannula 20. The needle cannula 20 further has a distal end with a distal tip 21 for penetrating the skin (S) of the user during injection and a proximal end 22 which is inserted into the cartridge 10.

The cartridge 10 is sealed at the distal end by a pierceable septum 11 whereas the proximal end of the cartridge 10 is provided with a non-shown plunger which can be moved in the distal direction by the dose engine to thereby pressurize the cartridge 10 such that a quantum of the preservative containing liquid drug inside the cartridge 10 is pressed through the lumen 23 of the needle cannula 20 and into the body of the user.

The needle cannula 20 is secured in a needle hub 30 which in the initiated state is clicked onto the housing structure 1 by a click-arm 31 which engages the housing structure 1. The needle cannula 20 is preferably glued to the needle hub 30 but could be secured in alternative ways.

Further, a needle shield 40 is provided which needle shield 40 is able to slide telescopically after being unlocked. As explained in WO 2017/032599, the needle shield 40 is prevented form axial movement in the initial state (FIG. 1) but can be unlocked by a rotation of the needle shield 40 into the NPR state. In the NPR state which is disclosed in FIG. 2 the needle shield 40 is able to slide axially in the proximal direction and an injection can be performed.

The injection is preferably done by the user pressing the distal end of the needle shield 40 against the skin “S” as indicated in FIG. 3. The axial movement of the needle shield 40 triggers the dose engine to release the torque in the torsion spring which thus moves the piston rod and the plunger inside the cartridge 10 forward to thereby pressurize the cartridge 10.

Distally the needle shield 40 carries a cleaning assembly 45 which comprises a cleaning chamber 46. The cleaning chamber 46 contains an amount of the same liquid drug as present in the cartridge 10 and is distally sealed by a pierceable septum 47. Proximally, the cleaning chamber 46 is closed by a movable plunger 48 which is able to move proximally as the cleaning chamber 46 is being filled with preservative containing liquid drug from the cartridge 10.

The cleaning chamber 46 also, in one example, comprises a narrow channel 49. Should the needle cannula 20 be accidentally bended during use then the needle cannula 20 will be straightened when the cleaning assembly 45 is moved distally after the injection has been performed. It is in that respect important that the channel 49 is narrow to fit the outer diameter of the needle cannula 20. However, the channel 49 can have a larger diameter if not functioning as a straightener. The movable plunger 48 which comprises a soft distal part doing the actually sealing of the cleaning chamber 46 and a more hard proximal part which is also provided with a narrow channel to assist in straightened the needle cannula 20 if bended.

Further, a needle cannula 20 which is especially suitable for use in the described cleaning assembly 45 is the needle cannula 20 described in International patent application No.: PCT/EP2019/061830 filed by Novo Nordisk NS, which is hereby included by reference. Especially, the grind on this needle cannula 20 hinders that the distal tip 21 of the needle cannula slices of fragments from the walls of the narrow channel 49 both during manufacturing of the injection device and in later use of the injection device. The distal tip 21 of the needle cannula 20 can further be glass blasted or electro polished to further prevent fragmentation of the walls of the channel 49.

The cleaning assembly 45 is both rotational and axial connected to the needle shield 40 such that the cleaning assembly 45 follow both axial and rotational movement of the needle shield 40.

In the NPR state disclosed in FIG. 2 the needle shield 40 has been rotated helically in the proximal direction such that the distal tip 21 of the needle cannula 20 is positioned outside the cleaning chamber 46. When the distal tip 21 of the needle cannula 20 is positioned outside the cleaning chamber 46, the pressure in the lumen 23 of the needle cannula 20 and inside the cartridge 10 can equalized with the atmospheric pressure surrounding the liquid system. The NPR state henceforth has the purpose of venting the liquid system before performing an injection. If a pressure is allowed in the interior of the cartridge 10 when the needle cannula 20 is inserted into the skin (S) of the user, such overpressure would pump out liquid drug and a wrongful amount of liquid drug would be injected. It is thus important to vent the liquid system prior to performing an injection. As seen in FIG. 2, the distal tip 21 of the needle cannula 20 is maintained protected by the needle shield 40 in this NPR position.

Connected to the hub 30 is a flexible arm 50 which has an inwardly protruding extension 51 which points towards the needle cannula 20 as depicted in FIG. 1. On the outer surface of the flexible arm 50, opposite the inwardly protruding extension 51, an outwardly pointing protrusion 52 is provided. Further, the flexible arm 50 has a distally pointing end surface 53.

In the initial state disclosed in FIG. 1, the flexible arm 50 is at rest and no radial force is applied onto the needle cannula 20.

The needle shield 40 is further provided with a raised locking area 41 on the inner surface. This raised locking area 41 is shaped as lump or protrusion and is provided with a sloped surface 42 pointing in the proximal direction when seen from the highest point.

When the user rotate the needle shield 40 from the initiated state of FIG. 1 and into the NPR state of FIG. 2, this raised locking area 41 follows the helical movement of the needle shield 40 and is thus moved helically. During this helical movement the sloped surface 42 engages with the outwardly pointing protrusion 52 on the flexible arm 50 and pushes the outwardly pointing protrusion 52 inwardly towards the centre line (X) as best seen in FIG. 2.

This engagement provides a radial force on the flexible arm 50 which thus bends inwardly and applies a radial force on the needle cannula 20. The radial force on the needle cannula (20) is preferably made by the inwardly protruding extension 51 and is indicated with the arrow (F) in FIG. 2. In this position, the end surface 53 of the flexible arm 50 is forced outwardly as best seen in FIG. 2 thus allowing the needle shield 40 to move axially.

During injection, the user pushes the needle shield 40 against the skin (S) as previously explained and further indicated in FIG. 3. The axial movement of the needle shield 40 activates the injection device to expel the set dose through the lumen 23 of the needle cannula 20.

During injection as disclosed in FIG. 3, the needle shield 40 is moved proximally and the raised locking area 41 follow this axial movement and thus releases the radial force on the protrusion 52 on the flexible arm 50. The result being that no radial force is applied onto the needle cannula 20 during injection. When the proper amount of preservative containing liquid drug has been injected into the body of the user, the user removes the distal end of the needle shield 40 from the skin and a non-shown spring automatically moves the needle shield 40 distally into the NPR position disclosed in FIG. 2.

In the NPR state of FIG. 2, the needle shield 40 is unlocked and free to move in the proximal direction. If the user, when the injection device is in the NPR state, accidentally drops the injection device onto a hard surface this could potentially damage the needle cannula 20. The distal tip 21 of the needle cannula 20 would thus together with the distal end of the needle shield 40 be forced in the proximal direction by the impact with the hard surface. This would course the needle cannula 20 to bend if the force of the impact is above the force usually required to bend the needle cannula. Such bending of the needle cannula 20 could potential obstruct the passage through the lumen 2. It is henceforth desired to somehow inform the user that the needle cannula 20 is inoperable.

The flexible arm 50 urging a radial force onto the needle cannula 20 will whenever the needle cannula 20 is bended encourage and amplify the needle cannula 20 to bend in a controlled manner in the radial direction following the radial force F applied onto the needle cannula 20. This is indicated by the arrow B in FIG. 4.

When the needle cannula 20 accidentally impacts the hard surface and starts to bend, the flexible arm 50 will not only amplify the bending but will also move further in the radial direction as disclosed in FIG. 4 such that the distal end 53 of the flexible arm 50 moves radially into a position in which the flexible arm 50 prevents axial movement of the cleaning assembly 45 and thus of the needle shield 40.

When the force applied onto the needle cannula 20 by the flexible arm 50 amplifies the bending of the needle cannula 20, the distal tip 21 also move in the proximal direction as the proximal end 22 of the needle cannula is anchored in the needle hub 30 and is unable to move. The distal tip 21 of the needle cannula 20 is thus moved into the needle shield 40 and cannot be used if the user should try to perform a new injection. At the same time the distal end 53 of the flexible arm 50 prevents the needle shield 40 from moving in the proximal direction. It is thus not possible for the user to perform an injection if the needle cannula 20 has been damaged.

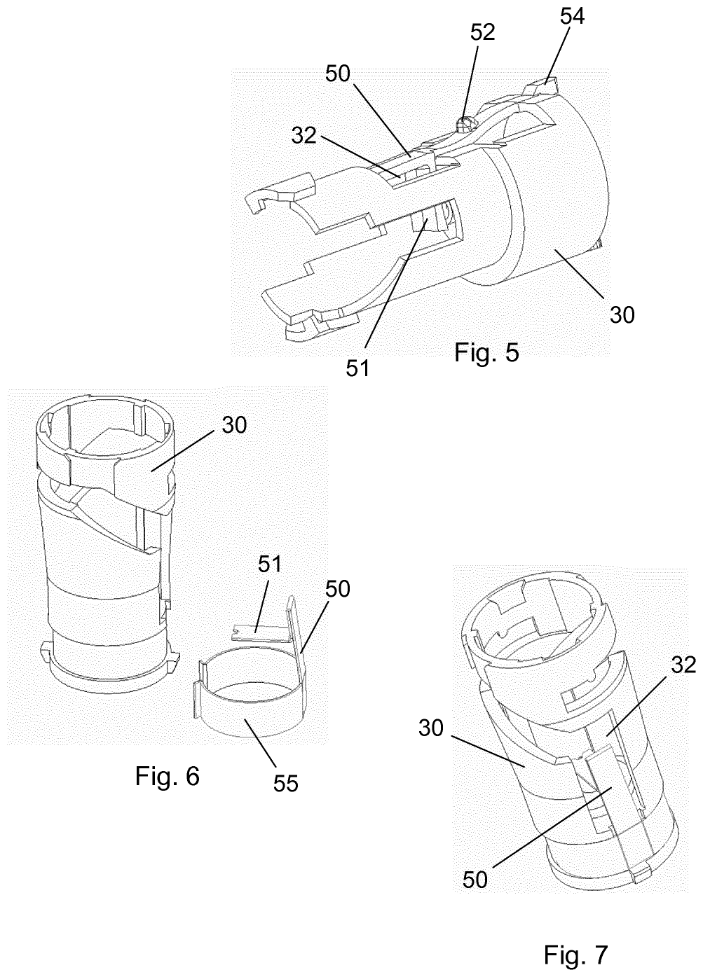

FIG. 5 discloses the flexible arm 50 attached to the needle hub 30. The flexible arm 50 can either be an integral part of the needle hub 30 or it can be a separate part connected to the needle hub 30. The proximal part of the flexible arm 50 is in one example be provided with a knob 54 engaging the housing structure 1 such that proximal end of the flexible arm 50 is secured between the housing structure 1 and the needle hub 30. In one example the flexible arm 50 is moulded from a suitable polymer.

Further, the needle hub 30 is preferably provided with an opening 32 through which opening 32 the flexible arm 50 operates when it is flexed radially.

A slightly different example of a flexible arm 50 is disclosed in FIG. 6 and in FIG. 7. Since the working principle of this second example is as in the first example; similar parts have been numbered with the same numbering.

The flexible arm 50 herein disclosed is formed from a metallic tape which proximally has two radial bands 55 which can be clicked onto the needle hub 30 such that the radial extension 51 extend through the opening 32 in the needle hub 30 as best seen in FIG. 7.

Some preferred embodiments have been shown in the foregoing, but it should be stressed that the invention is not limited to these, but may be embodied in other ways within the subject matter defined in the following claims.

1. An injection device for injecting doses of a liquid drug, comprising:
a housing structure holding a container containing the liquid drug to be injected,
a needle cannula connected to the housing structure and having a distal end with a distal tip for penetrating the skin (S) of a user and a proximal end for connecting to the container and a lumen there between,
a movable needle shield movable relatively to the housing structure between a first position and a second position;
the first position being a position in which the needle shield covers at least the distal tip of the needle cannula,
the second position being a position in which the needle shield is retracted such that the distal tip of the needle cannula is exposed,
and wherein the injection device further comprises an immobilization mechanism which immobilizes the injection device in response to the needle cannula being exposed to an axial force above a predetermined value and which immobilization mechanism comprises a flexible arm which apply a radial force onto the needle cannula.

2. An injection device according to claim 1, wherein the predetermined value is the axial force needed to bend the needle cannula.

3. An injection device according to claim 1, wherein the flexible arm is movable from a first radial position to a second radial position in response to bending of the needle cannula.

4. An injection device according to claim 3, wherein the flexible arm when in the second radial position prevents axial movement of the needle shield.

5. An injection device according to claim 3, wherein the flexible arm is provided with a distally pointing end surface which engages the needle shield when the flexible arm is in the second radial position.

6. An injection device according to claim 1, wherein the flexible arm is connected to a needle hub securing the needle cannula.

7. An injection device according to claim 1, wherein a radial force urges the flexible arm radially against the needle cannula.

8. An injection device according to claim 1, wherein the flexible arm is provided with an outwardly pointing protrusion.

9. An injection device according to claim 1, wherein the needle shield is rotatable relatively to the housing structure from a locked to an unlocked position.

10. An injection device according to claim 9, wherein the protrusion abuts the needle shield when the needle shield is rotated to the unlocked position.

11. An injection device according to claim 1, wherein the needle shield is provided with a raised locking area on an inner surface thereof.

12. An injection device according to claim 11, wherein the abutment of the raised locking area with the protrusion generates the radial force urging the flexible arm radially against the needle cannula.

13. An injection device according to claim 11, wherein the raised locking area is provided with at least one sloped surface.

14. An injection device according to claim 1, wherein the flexible arm has a radial extension abutting the needle cannula.
